(19) United States
(12) Patent Application Publication
(10) Pub. No.: US 2003/0176825 A1
(43) Pub. Date: Sep. 18, 2003

(54) Adjustable, elastic fixation device

(75) Inventor: Amotz Yavnai, Kiryat Motzkin (IL)

Correspondence Address: G.E. EHRLICH (1995) LTD., c/o ANTHONY CASTORINA, 2001 JEFFERSON DAVIS HIGHWAY, SUITE 207, ARLINGTON, VA, 22202, US

(73) Assignee: Ortech Innovative Orthopedic Devices, Ltd.

(21) Appl. No.: 10/098,540

(22) Filed: Mar. 18, 2002

(51) Int. Cl. A61F 5/00

(52) U.S. Cl. 602/13; 602/60; 602/75; 602/3

(57) ABSTRACT

An inflatable, elastic fixation device is provided for adjustable orthopedic fixation. The device is adapted for fastening onto a section of a body, to a desired clearance; and inflating to desired levels of stiffness and tightness. Adjustments may be made when the device is first applied, as well as with time and with activity. The fixation device may be removed for washing or treatment and reapplied. When inflated, the device is rigid, and operative as a structural element for redistributing loads to lessen a pressure on the section of body or to prevent it.

### FIELD OF THE INVENTION

The present invention relates generally to orthopedic fixation, and in particular, to a device and method for adjustable fixation.

### BACKGROUND OF THE INVENTION

Orthopedic fixation, for example, for immobilizing a broken bone, an arthritic joint, or part or all of the spine, often relies on a rigid dressing, made of gauze and plaster of Paris, know as a plaster cast.

The plaster cast has several disadvantages:

1. when applied over a swollen section of the body, and the swelling is reduced, a clearance forms between the cast and the section of the body;

2. it is weighty;

3. it may press unevenly on the body;

4. generally, it does not redistribute loads away from the portion of the body under fixation

5. it cannot be removed for washing; and

6. once applied, skin irritations under the cast cannot be treated.

Additionally, there are situations, for example, a fracture of a rib, or lower back pain, which cannot be treated with a plaster cast.

U.S. Pat. No. 5,704,904 to Dunfee describes an inflatable lumbar traction cast, capable of preventing spinal injuries and of facilitating healing of existing injuries. U.S. Pat. No. 5,980,560 to Chang describes an air-injection stomach band for stretching and (or) extending the lumbar vertebra of patients of dislocated discs. However, these devices are applicable only for the lower back and do not offer a general solution to orthopedic fixation.

There is thus a widely recognized need for, and it would be highly advantageous to have, a system and method for orthopedic fixation, devoid of the above limitations.

### SUMMARY OF THE INVENTION

In accordance with an aspect of the present invention, there is thus provided an adjustable, elastic fixation device, comprising:

inner and outer thin, flexible, airtight materials, designed to substantially superimpose;

airtight seams, connecting edges of the inner and outer airtight materials, defining an inflatable volume within;

a flexible structure, arranged between the inner and outer materials, for providing a generally even thickness to the inflatable volume, in the inflated state;

a valve, in fluid communication with the inflatable volume, for selectably inflating and selectably deflating the device, as desired;

a controlled inflation device, which may be selectably mounted on the valve, and selectably detached from the valve, for providing gradual inflation to the inflatable volume; and

an adjustable fastener, for adjustably fastening the device on a section of a body.

In accordance with an additional aspect of the present invention, the device further includes at least one pressure gauge, for monitoring at least one pressure selected from the group consisting of the pressure within the inflatable volume and the pressure between the device and the body. Additionally, both the pressure within the inflatable volume and the pressure between the device and the body may be monitored.

In accordance with an additional aspect of the present invention, when unfastened, the device may open to a generally flat configuration.

In accordance with yet an additional aspect of the present invention, the device is formed as a sleeve.

In accordance with still an additional aspect of the present invention, the device includes a replaceable interface between the inner material, designed on a side of the body, and the body, the replaceable interface being selected from the group consisting of a washable interface and a disposable interface.

In accordance with another aspect of the present invention, the device further includes a wrapper, which wraps said inner and outer materials.

In accordance with an additional aspect of the present invention, the wrapper is replaceable.

In accordance with still an additional aspect of the present invention, the flexible structure includes an elastic structure, formed of first and second elastic layers which define repetitive cutout structures thereon, and which are connected by fiber columns.

In accordance with another aspect of the present invention, the flexible structure includes an elastic structure, formed of coiled springs.

In accordance with still another aspect of the present invention, the flexible structure includes an interconnected channel structure.

In accordance with an additional aspect of the present invention, the adjustable fastener includes at least one pair of fastening strips, designed along a length of the device, parallel to a length axis of the body.

In accordance with another aspect of the present invention, the adjustable fastener includes at least two pairs of fastening strips, designed along a width of the device, perpendicular to a length axis of the body.

In accordance with still another aspect of the present invention, the adjustable fastener includes at least two drawstrings, designed within respective draw channels.

In accordance with an additional aspect of the present invention, the device includes a plurality of sections, selected from the group consisting of length and width sections, each adapted to be inflated individually.

In accordance with still an additional aspect of the present invention, each section is adapted to be fastened individually.

In accordance with an additional aspect of the present invention, the device is designed as a belt.

In accordance with another aspect of the present invention, the device is designed as a neck support.

In accordance with still another aspect of the present invention, the device is designed as an arm support.

In accordance with yet another aspect of the present invention, the device is designed as a leg support.

In accordance with still another aspect of the present invention, the device is designed as a knee support.

In accordance with yet another aspect of the present invention, the device is designed as an ankle support.

In accordance with still another aspect of the present invention, the device is designed as a hip-joint support.

In accordance with yet another aspect of the present invention, the device is designed as an interface between a body stump and an artificial limb.

In accordance with still another aspect of the present invention, the device is designed as an interface between a body and a backpack.

In accordance with yet another aspect of the present invention, the device is designed as an interface between a body and a child carrier.

In accordance with still another aspect of the present invention, the device is designed as a bullet-proof vest, or a part thereof, or an interface for a bullet-proof vest.

In accordance with still another aspect of the present invention, the device is designed as a combat vest, or a part thereof, or an interface for a combat vest.

In accordance with yet another aspect of the present invention, the device is designed as an outdoor vest, and further includes pockets.

In accordance with still another aspect of the present invention, the device is designed as a fishing vest and further includes a fishing poll holder.

In accordance with an aspect of the present invention, there is thus provided a method of orthopedic fixation, comprising:

selecting an adjustable, elastic fixation device, of a proper size and mode of fixation, the device comprising:

inner and outer thin, flexible, airtight materials, designed to substantially superimpose;

airtight seams, connecting edges of the inner and outer airtight materials, defining an inflatable volume within;

a flexible structure, arranged between the inner and outer materials, for providing a generally even thickness to the inflatable volume, in the inflated state;

a valve, in fluid communication with the inflatable volume, for selectably inflating and selectably deflating the device, as desired;

a controlled inflation device, which may be selectably mounted on the valve, and selectably detached from the valve, for providing gradual inflation to the inflatable volume; and

an adjustable fastener, for adjustably fastening the device on a section of a body;

fastening the device onto a section of a body, to the desired clearance; and

inflating the inflatable volume, to desired levels of stiffness and tightness.

In accordance with an additional aspect of the present invention, the method includes correcting the fastening after the inflating, to correct said desired levels of stiffness and tightness.

In accordance with yet an additional aspect of the present invention, the method includes adjusting the fastening with time.

In accordance with still an additional aspect of the present invention, the method includes adjusting the fastening with activity.

In accordance with yet an additional aspect of the present invention, the method includes adjusting the inflation with time.

In accordance with still an additional aspect of the present invention, the method includes adjusting the inflation with activity.

In accordance with yet an additional aspect of the present invention, the method includes removing and reapplying the device, as needed.

The present invention is of an inflatable, elastic fixation device for adjustable orthopedic fixation. The device is adapted for fastening onto a section of a body, to a desired clearance; and inflating to desired levels of stiffness and tightness. Adjustments may be made when the device is first applied, as well as with time and with activity. The fixation device may be removed for washing or treatment and reapplied. When inflated, the device is rigid, and operative as a structural element for redistributing loads to lessen a pressure on the section of body or to prevent it.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1L schematically illustrate adjustable, elastic fixation devices, in accordance with preferred embodiments of the present invention;

FIGS. 2A-2F schematically illustrate adjustable, elastic fixation devices, in accordance with other preferred embodiments of the present invention.

FIGS. 3A-3D schematically illustrate an adjustable, elastic fixation device, in accordance with another preferred embodiment of the present invention;

FIG. 4 schematically illustrates an adjustable, elastic fixation device, designed as a wide belt, in accordance with a preferred embodiment of the present invention;

FIG. 5 schematically illustrates an adjustable, elastic fixation device, adapted for hip-joint fixation, in accordance with another preferred embodiment of the present invention;

FIG. 6 schematically illustrates an adjustable, elastic fixation device, adapted for neck fixation, in accordance with yet another preferred embodiment of the present invention;

FIG. 7 schematically illustrates an adjustable, elastic fixation device, adapted for arm fixation, in accordance with still another preferred embodiment of the present invention;

FIG. 8 schematically illustrates an adjustable, elastic fixation device, adapted for ankle fixation, in accordance with yet another preferred embodiment of the present invention;

FIG. 9 schematically illustrates an adjustable, elastic fixation device, adapted for knee fixation, in accordance with still another preferred embodiment of the present invention;

FIG. 10 schematically illustrates an adjustable, elastic fixation device, adapted for leg fixation, in accordance with yet another preferred embodiment of the present invention;

FIG. 11 schematically illustrates an adjustable, elastic fixation device, adapted for shoulder fixation, in accordance with still another preferred embodiment of the present invention;

FIG. 12 schematically illustrates an adjustable, elastic fixation device, designed as an interface between an artificial limb and a stump, in accordance with still another preferred embodiment of the present invention;

FIGS. 13A-13C schematically illustrate adjustable, elastic fixation devices designed as vests, in accordance with yet another preferred embodiment of the present invention;

FIG. 14 schematically illustrates an adjustable, elastic fixation device designed as an interface between a body and a backpack, in accordance with still another preferred embodiment of the present invention; and

FIG. 15 schematically illustrates an adjustable, elastic fixation device designed as an interface between a body and a baby carrier, in accordance with still another preferred embodiment of the present invention.

### DESCRIPTION OF THE PREFERRED EMBODIMENTS

The present invention is of an inflatable, elastic fixation device for adjustable orthopedic fixation. Specifically, the present invention provides for a device, which is adapted for fastening onto a section of a body, to a desired clearance; and inflating to desired levels of stiffness and tightness. Adjustments may be made when the device is first applied, as well as with time and with activity. The fixation device may be removed for washing or treatment and reapplied. When inflated, the device is rigid, and operative as a structural element for redistributing loads to lessen a pressure on the section of body or to prevent it.

The principles and operation of the systems and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

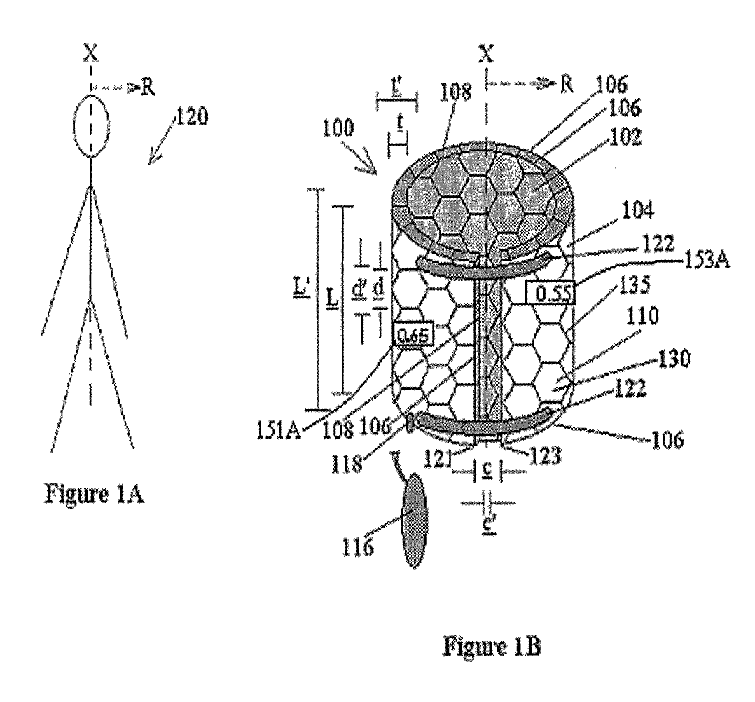

Referring now to the drawings, FIGS. 1A-1G schematically illustrate an adjustable, elastic fixation device 100, in accordance with a preferred embodiment of the present invention. As seen in FIG. 1A, a body 120, defines a radial coordinate system, X;R, used in the description of device 100. However, although the present invention is illustrated vis a vis a human, it will be appreciated that it is applicable also to animals.

As seen in FIG. 1B, representing a pictorial view, device 100 includes inner and outer thin, airtight materials 102 and 104, of substantially equal dimensions, so as to substantially superimpose. In the uninflated state, materials 102 and 104 have a length L, along the X axis, wherein length L is preselected based generally on the length of the body section for fixation. Additionally, an airtight edge strip 108, may be used between materials 102 and 104. Edge strip 108 has a thickness t, in the uninflated state.

Airtight edge strip 108 and airtight inner and outer materials 102 and 104 are formed of preferably, lightweight and preferably, elastic materials, such as, polyester, polyurethane, nylon, PVC, natural or synthetic rubber, and the like, or a blend thereof, for example, a blend of polyurethane and nylon. They may all be formed of the same material, or of different materials, which may further have different elasticity.

Airtight seams 106 connect edge strip 108 and materials 102 and 104, thus defining an inflatable volume within.

Airtight seams 106 may be bonded with glue, joined by heat and pressure, or both. Alternatively, another method that produces an airtight seam may be used.

Furthermore, device 100 includes a controlled inflation device 116, for gradual inflation of the inflatable volume, and a valve 118. Preferably, controlled inflation device 116 is designed to selectably communicate with valve 118 for inflating device 100 and to selectably detach from valve 118 after inflation. Alternatively, controlled inflation device 116 may be permanently attached to device 100. Controlled inflation device 116 may be a manual pump, such as a hand or a foot pump, or an electric pump, which may be battery operated or powered from a grid. Alternatively, inflation device 116 may be a liquefied gas canister, or a gas generator. The inflating gas may be air, nitrogen, carbon dioxide, helium or any other noncombustible, preferably, environmentally friendly gas. Preferably, electric controlled inflation device 116 may also be powered by a car battery. Valve 118 may further be used for deflating device 100, when desired. Additionally, valve 118 may be pushed inside the inflatable volume so as to be flush with outer material 104, when not in used.

Additionally, device 100 may include a first pressure gauge, for allowing a user to monitor gas pressure within the inflatable volume.

Preferably, the first pressure gauge includes a first display 151A, for example, a first digital display 151A, external to outer material 104 and in signal communication with a first pressure sensor 151B (not seen in FIG. 1B, but seen in FIGS. 1D-1G, hereinbelow). First pressure sensor 151B is designed inside the inflatable volume. Additionally, a plurality of first pressure gauges may be used.

Furthermore, device 100 may include a second pressure gauge, for allowing a user to monitor the pressure between device 100 and body 120. Preferably, the second pressure gauge includes a second display 153A, for example, a second digital display 153A, external to outer material 104 and in signal communication with a second pressure sensor 153B (not seen in FIG. 1B, but seen in FIGS. 1D-1G, hereinbelow). Second pressure sensor 153B is arranged between inner material 102 and body 120. Moreover, a plurality of second pressure gauges may be used.

Additionally, device 100 includes an adjustable fastener 122, for example, at least two pairs of straps 122, designed along the circumference of device 100, for fastening device 100 to a section of body 120. The fastening mechanism of each pair of straps 122 may be any one of a buckle, snaps, Velcro strips, and the like. Thus, straps 122 may be used to adjustably fasten device 100 to body 120. Preferably, in the uninflated state, a closure c, of preferably up to about 6 centimeters, remains between edges 121 and 123 of device 100.

When inflated, device 100 expands in three directions, extending along the X and R axes, and along its circumference. Thus, uninflated length L extends to an inflated length L′, uninflated height t extends to an inflated height t′, uninflated size d extends to an inflated size d′, and uninflated closure c between edges 121 and 123 is reduced to an inflated closure c′

Device 100 further includes a flexible inner structure 110, arranged between materials 102 and 104 (as described in detail in FIGS. 1C-1E, hereinbelow), for providing generally even inflation to the inflatable volume.

As seen in FIG. 1C, flexible structure 110 may includes an elastic structure 130, having first and second elastic layers 132 and 134, preferably formed as woven or knotted elastic fiber meshes. Alternatively, first and second elastic layers 132 and 134 may be formed as an elastic layer of rubber, PVC, and the like. Preferably, a thickness e of layer 132 and a thickness f of layer 134, are about 1 mm, each.

Additionally, first and second elastic layers 132 and 134 define repetitive cutout structures 131 and 133, respectively, such as a repetitive polygonal cutout structure, for example, of hexagons, diamonds, squares, triangles and the like, or a repetitive circular, elliptical or semicircular cutout structure, or the like. Preferably, a size d of the cutout is between about 0.3 and about 1 cm.

First and second layers 132 and 134 are connected by preferably elastic fiber columns 135. Preferably, an overall height of elastic structure 130 is t, substantially matching the thickness of edge strip 108 (FIG. 1B). Preferably, t is between 0.5 and 1 cm.

It will be appreciated that other dimensions are similarly possible in the construction of elastic structure 130 and device 100.

The inflation of device 100 is further illustrated in FIGS. 1D-1E, which are cross-sectional views of device 110, in the uninflated and inflated states, respectively, when unfastened. Additionally, FIGS. 1D and 1E illustrate the specific advantage of inner structure 110, formed as elastic structure 130 (FIG. 1C)

Inner material 102 is bonded onto an external surface of first layer 132, and outer material 104 is bound to an external surface of second layer 134. Repetitive cutout structures 131 and 133 thus form imprints on inner and outer materials 102 and 104, as seen in FIG. 1B for outer material 104.

As seen in FIG. 1D, in the uninflated state, device 100 is characterized by a width W, which is preselected generally based on the circumference of the body section for fixation. Inner structure 110 is characterized by height t and size d.

As seen in FIG. 1E, in the inflated state, device 100 is characterized by an inflated width W′, larger then W and elastic structure 130 is characterized by inflated height t′ and inflated size d′.

The specific advantage of inner structure 110, formed as elastic structure 130 (FIG. 1C) may be appreciated from FIGS. 1D and 1E. Because inner and outer materials 102 and 104 are bonded to the external surfaces of elastic structure 130, the inflation of these materials is constrained to areas over cutout sections 131 and 133, so as to form inflated domes over these cutout sections (FIG. 1E). By thus constraining the inflation, elastic structure 130 is operative to provide generally even inflation to the inflatable volume of device 100.

FIGS. 1D and 1E further illustrate the first pressure gauge, for allowing a user to monitor gas pressure within the inflatable volume, formed as first display 151A, external to outer material 104, and first pressure sensor 151B, designed inside the inflatable volume.

Furthermore, FIGS. 1D and 1E illustrate the second pressure gauge, for allowing a user to monitor pressure between inner material 102, and body 120, formed as second display 153A, external to outer material 104, and second pressure sensor 151B, arranged between inner material 102 and body 120.

The inflation of device 100 is still further illustrated in FIGS. 1F-1G, which are cross-sectional views of device 110, in the uninflated and inflated states, respectively, when fastened.

As seen in FIG. 1F, in the uninflated state, after fastening, there is a clearance u, between inner material 102 and body 120.

As seen in FIG. 1G, in the inflated state, inner material 102 presses against body 120, and closure c′ between edges 121 and 123 is reduced, compared to closure c (FIG. 1B).

FIGS. 1F and 1G further illustrate the first pressure gauge, for allowing a user to monitor gas pressure within the inflatable volume, formed as first display 151A, external to outer material 104, and first pressure sensor 151B, designed inside the inflatable volume.

Furthermore, FIGS. 1F and 1G illustrate the second pressure gauge, for allowing a user to monitor pressure between inner material 102, and body 120, formed as second display 153A, external to outer material 104, and second pressure sensor 151B, arranged between inner material 102 and body 120.

Preferably, device 100 is inflated to a relative pressure of between 0.1 and 0.7 bar, which causes length L to extend by preferably between 110 and 140%, and causes height t to extend by preferably between 110 and 200%. These extensions apply tensile stresses to body 120, along the X direction, and evenly distributed normal stresses along the R direction. The evenly distributed normal stresses are maintained at a comfortable range, by adjustments to fastener 122.

The tensile and normal stresses, applied by device 100 to body 120, are of significance in orthopedic fixation, specifically in bone fracture unification. The tensile stresses prevent the fracture edges from overlapping, while the normal stresses, pressing against the body, prevent movement of the bone fracture.

Additionally, where device 100 is applied to a lower back, it transfers loads from the spinal column to the pelvis, since when inflated, device 100 is rigid, and is operative as a structural element to redistribute loads, much like a truss element.

In accordance with the present invention, a range of sizes is provided for each mode of fixation, such as fixation of a lower back, an arm, or a neck.

Further in accordance with the present invention, the method of fixation by device 100 includes:

selecting device 100 of a proper size and mode of fixation;

fastening device 100 on a section of body 120, to a desired uninflated clearance u (FIG. 1F); and

inflating device 100 to desired levels of stiffness and tightness, wherein the stiffness is determined by the pressure within the inflated volume, and the tightness is determined by the normal stresses, pressing against body 120.

It will be appreciated that there is interplay between the tightness and the stiffness. Thus, it may be necessary to further adjust straps 122, to arrive at a comfortable combination of tightness and stiffness.

The method may further include correcting the tightness with time, for example, tightening straps 122 as a swelling is reduced. Additionally, the method may include varying the tightness and stiffness between activities, for example, between resting and working. Furthermore, the method may include removing device 100, for washing body 120, for dressing wounds, or for treating skin irritations, then reapplying device 100. Additionally, the method may include removing device 100, to facilitate its washing.

It will be appreciated that device 100 is advantageous over a plaster cast, and other known fixating devices, for the following reasons:

As a swollen section of the body is fixated, and the swelling is reduced, a clearance between the fixation and the body develops. With a plaster cast, there is no remedy for this situation. Yet, the tightness of device 100 may be readjusted, by adjusting fastener 122 and (or) by adjusting the inflation, as necessary.

Furthermore, while a plaster cast has a single stiffness, device 100 may be applied with varying stiffness. For example, a high stiffness, which substantially matches that of a plaster cast, when body 120 engages in activities, and a reduced stiffness, when body 120 rests.

Additionally, unlike a plaster cast, which cannot be removed once applied, device 100 may be removed and reapplied easily, as has been noted hereinabove.

Unlike U.S. Pat. No. 5,724,993, incorporated herein by reference, which includes only several inflatable columns around the circumference of body 120 (FIG. 1A), device 100 is operative as a rigid element, continuously, around the circumference of body 120. Additionally, device 100 applies comfortable, evenly distributed normal stresses in the R direction to body 120. These normal stresses are important for fracture unification, as they prevent movement of the bone fractures. Additionally, they are important for back fixation, by minimizing possible movement of the vertebrae and ribs, and by reducing back pain.

Referring further to the drawings, FIGS. 1H-1L schematically illustrate adjustable, elastic fixation devices 100, in accordance with other preferred embodiments of the present invention.

As seen in FIG. 1H, inner and outer materials 102 and 104 may be different materials and have different elasticity. Thus, while inner material 102 may elastically expand, so as to apply normal stresses on body 120, outer material 104 may not expand, or expand little.

As seen in FIG. 1I, device 100 may further include a preferably removable interface 109, for providing a material that is soft to the touch between body 120 and inner surface 102, and for preferably providing a fresh surface against body 120, clean of sweat, blood stains, and the like. Interface 109 may be washable or disposable, and may be attached to device 100 by any one of Velcro strips, snaps, and the like. Interface 109 may be formed, for example of cotton, a blend of cotton and polyester, paper, or the like. Alternatively, interface 109 is not removable.

Additionally or alternatively, as seen in FIG. 1J, device 100 may further include a wrapper 103 similar to a pillow case, for wrapping device 100. Wrapper 103 may further be removable, and may be washable or disposable. Wrapper 103 provides a material that is soft to the touch and may be formed, for example of cotton, a blend of cotton and polyester, paper or the like.

As seen in FIG. 1K, elastic structure 130 may be formed as a plurality of coiled springs 142, each designed with the coil around the R axis, wherein inner and outer materials 102 and 104 are bonded to upper and lower edges 144 and 146 of the coils, forming circular imprints on inner and outer materials 102 and 104. The coiled springs may be formed of metal or alloy, for example, stainless steel. Alternatively, they may be formed of a resilient plastic, or another suitable material.

As seen in FIG. 1L, inner material 102 may be somewhat larger than outer material 104, and include cutout corners, having edges 107A and 107B, forming margins 105, having edges 101. Outer material 104 includes edges 111.

To form the inflatable volume, edges 107A and 107B of inner material 102 are joined by airtight seams 106 (FIG. 1B), to create a box-like structure, and edges 101 of inner material 102 are joined with edges 111 of outer material 104 by airtight seams 106. Alternatively, outer material 104 may be somewhat larger than inner material 102.

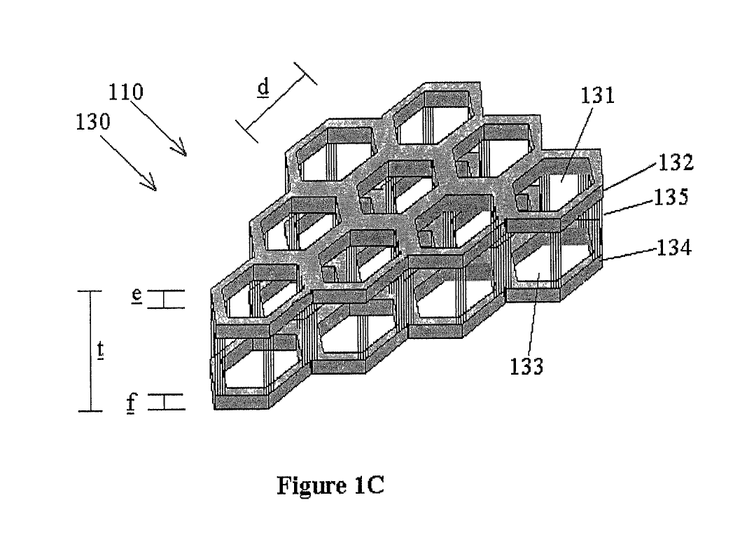

It will be appreciated, that when unfastened, device 100 opens to a generally flat configuration, as shown in FIG. 1D and is further illustrated hereinbelow, in conjunction with FIGS. 2C-2D. In its generally flat configuration, device 100 may be slid under an injured person, for example at an accident scene, to fixate the back, the neck, or other parts of the body of the injured person directly at the accident scene, before transportation or movement.

It will be appreciated that device 100 may also be designed for whole body fixation, for example in accident conditions.

Referring further to the drawings, FIGS. 2A-2F schematically illustrate adjustable, elastic fixation devices 100, in accordance with other preferred embodiments of the present invention.

As seen in FIGS. 2A-2C, device 100 includes inner and outer materials 102 and 104, of length L and width W and of inflated length L′ and width W′. Additionally, device 100 includes seams 106, bonding edges of inner and outer materials 102 and 104 to each other, to define the inflatable volume within. Furthermore, device 100 includes adjustable fasteners 122, formed for example, as straps 122 for fastening device 100 onto body 120.

In accordance with the present embodiment, flexible structure 110, which provides a generally even thickness to the inflatable volume, includes interconnected channels 110. The channels may be formed by joining inner and outer materials 102 and 104 with seams 125, at predetermined distances v (FIG. 2C), of preferably between 1 and 3 cm. Seams 125 may be bonded with glue, joined by heat and pressure, or both. Alternatively, seams 125 may be formed by another method, as known. Preferably, seams 125 are airtight.

Additionally, as seen in FIGS. 2A-2C, interconnected channels 110 may be formed as a plurality of length channels 124 and at least one, and preferably two width channels 126, preferably adjacent to seams 106, at the upper and lower edges of device 100, connecting between them. As seen in FIG. 2B, when inflated, channels 124 and 126 expand to inflated height t′, running in the R direction. Inflated width W′ may be smaller, about the same, or greater than width W, depending on the elasticity of materials 102 and 104. Similarly, an inflated distance v′ may be smaller, about the same, or greater than distance v (FIG. 2C). Preferably device 100 of the present embodiment is also inflated to a relative pressure of between 0.1 and 0.7 bars.

In accordance with another embodiment of the present invention, seen in FIG. 2D, seams 125 may be broken, to form length channels 127. FIG. 2D further illustrates adjustable fastener 122 as a single pair of adjustable Velcro strips 122, designed along the length of device 100. Adjustments may be made by the extent of overlap between Velcro strips 122.

It will be appreciated other ways of forming interconnected channels 110 are possible.

In accordance with yet another preferred embodiment of the present invention, seen in FIG. 2E, device 100 may be formed of a plurality of width sections, such as sections 112, 113, and 114, of individual lengths L1, L2, and L3, respectively, each being adapted for individual inflation, and having valve 118. Additionally, each section may have at least one fastener 122. The purpose of the plurality of sections is to provide for different stiffness and tightness along the length of device 100, for example, in cases of curvature of the spinal column. Additionally, the purpose of the plurality of sections is to adjust to the hourglass shape of the body, from below the waistline to above the waistline.

Additionally, each width section may include first pressure display 151A and first pressure sensor 151B. Furthermore, each width section may include second pressure display 153A and second pressure sensor 153B.

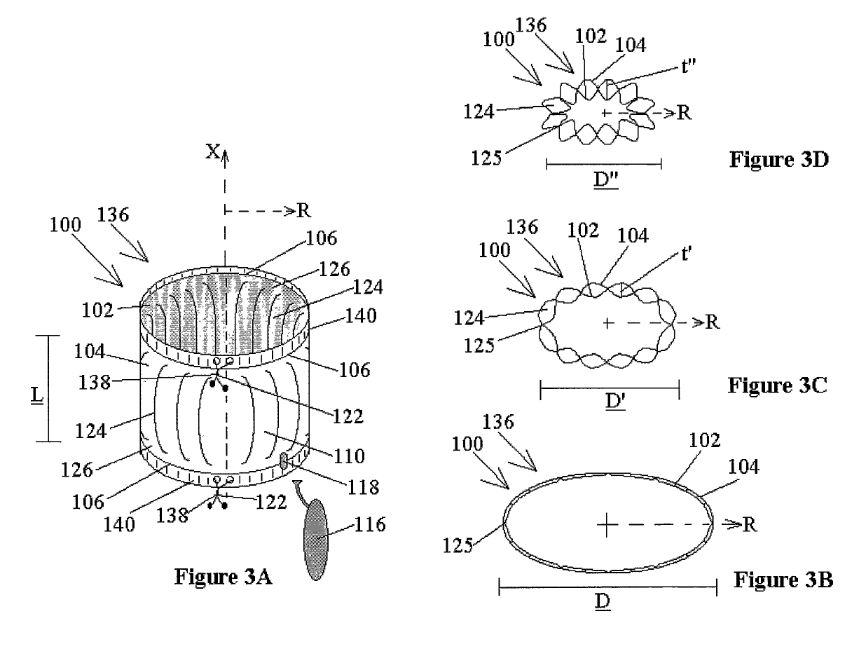

In accordance with still another embodiment of the present invention, seen in FIG. 2F, device 100 may be formed of a plurality of length sections, such as sections 141, 143, and 145, each adapted for individual inflation, and having valve 118. Thus, only a section of the body, for example a shoulder, illustrated hereinbelow, in conjunction with FIG. 11, may be fixated.

Additionally, each length section may include first pressure display 151A and first pressure sensor 151B. Furthermore, each length section may include second pressure display 153A and second pressure sensor 153B.

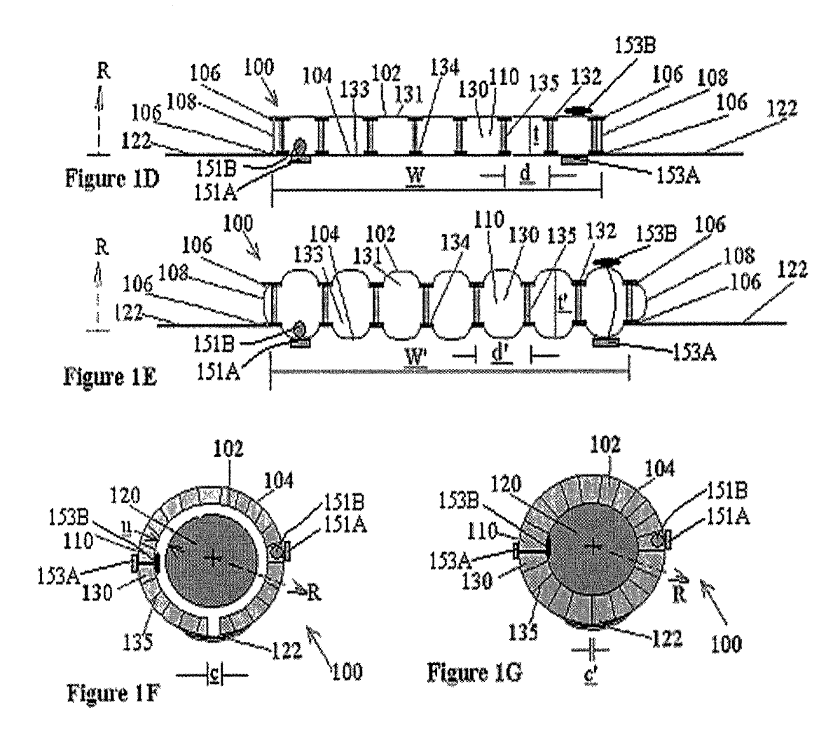

Referring further to the figures, FIGS. 3A-3D schematically illustrate adjustable, elastic fixation device 100, in accordance with still another preferred embodiment of the present invention.

As seen in FIGS. 3A-3B, device 100 includes inner and outer materials 102 and 104, of length L. Additionally, device 100 includes seams 106, bonding edges of inner and outer materials 102 and 104 to each other, to define the inflatable volume within. Furthermore, device 100 includes flexible structure 110, preferably formed as length channels 124, and at least one, and preferably two width channels 126, adjacent to seams 106, at the upper and lower edges of device 100, connecting between them.

In accordance with the present embodiment, materials 102 and 104 form a sleeve 136 of a diameter D. Additionally, in accordance with the present embodiment, adjustable fasteners 122 are formed as at least two drawstrings 138, designed within draw channels 140, which are not in fluid communication with channels 124 and 126. Rather draw channels 140 are sealed from channels 124 and 126 by seams 106.

Diameter D is larger than the required diameter for fixation. As seen in FIGS. 3C, draw strings 138 may be used to adjust the diameter for fixation to a diameter D′, smaller than D. When inflated, channels 124 and 126 expand to an inflated height t′, running in the R direction.

Additionally, as seen in FIGS. 3D, draw strings 138 may be used to adjust the diameter for fixation to a diameter D″, smaller than D and smaller than D′. When inflated, channels 124 and 126 expand to an inflated height t″, greater than t′, running in the R direction.

Thus, it will be appreciated that sleeve 136 may be adjusted to different fixation diameters.

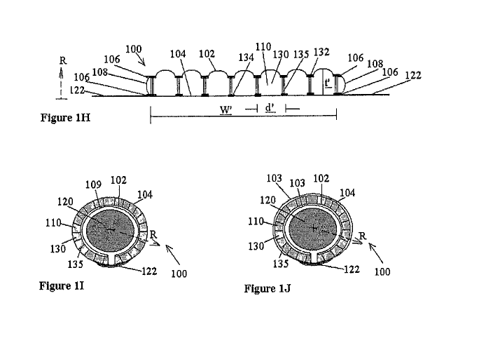

Referring further to the drawings, FIGS. 4-16 illustrate the use of adjustable, elastic fixation device 100 in various modes of orthopedic fixation, in accordance with preferred embodiments of the present invention.

As seen in FIG. 4, device 100 may be designed as a wide belt, for example, for the fixation of the lower back and (or) ribs.

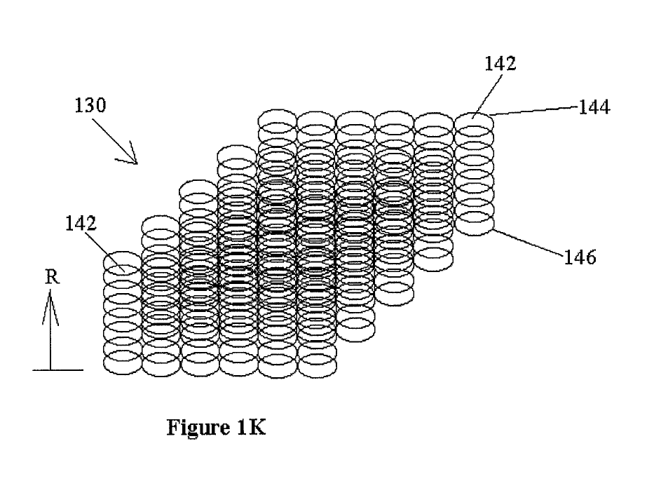

As seen in FIG. 5, device 100 may be adapted for the fixation of the hip joint. Adjustable fastener 122 is designed as a single pair of adjustable Velcro strips 122, similar to that shown in FIG. 2D. Additionally, device 100 may include drawstrings 138, designed within draw channels 140, around the hip. It will be appreciated that a similar structure, designed on both sides, may be used for the fixation of the pelvis.

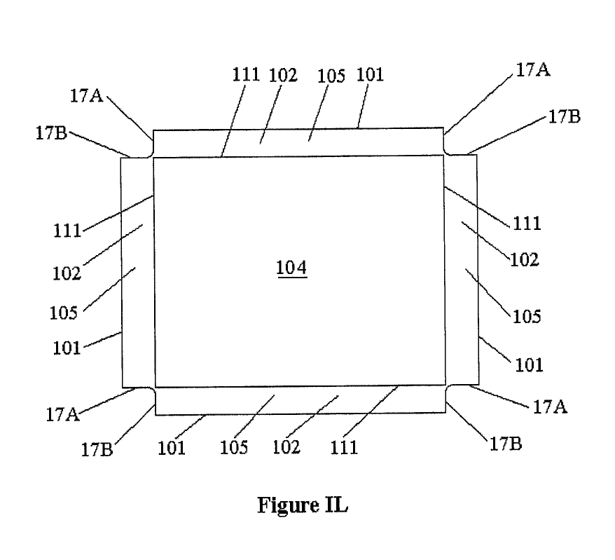

As seen in FIG. 6, device 100 may be adapted for the fixation of the neck.

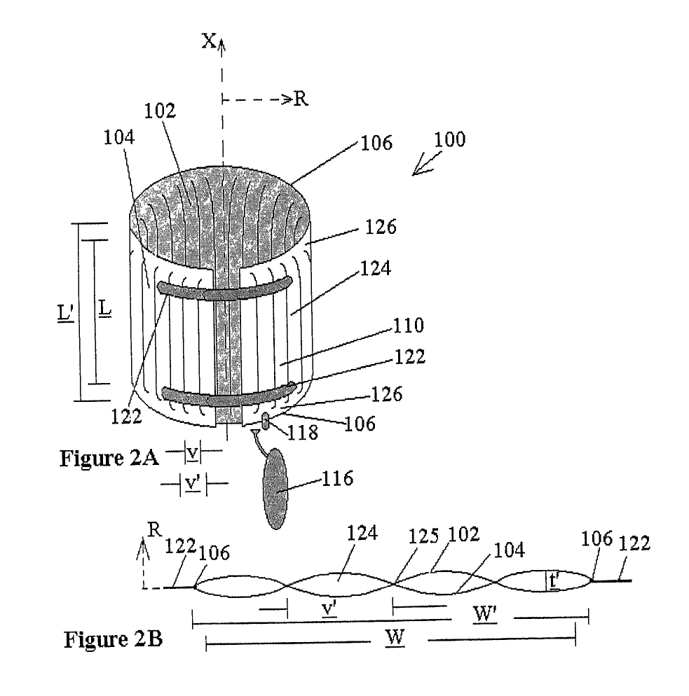

As seen in FIG. 7, device 100 may be adapted for the fixation of an arm.

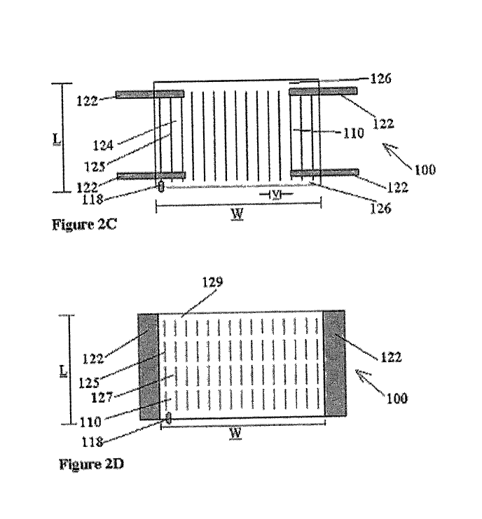

As seen in FIG. 8, device 100 may be adapted for the fixation of an ankle. A heel 115 may be attached to this configuration, for stepping. It should be noted that upon recovering from a sprain, device 100 may be used to gradually load the sprained area, by gradually by reducing the pressure. The first pressure gauge, formed of first pressure display 151A (FIG. 1D) and first pressure sensor 151B, are important in this configuration for gradually loading the ankle.

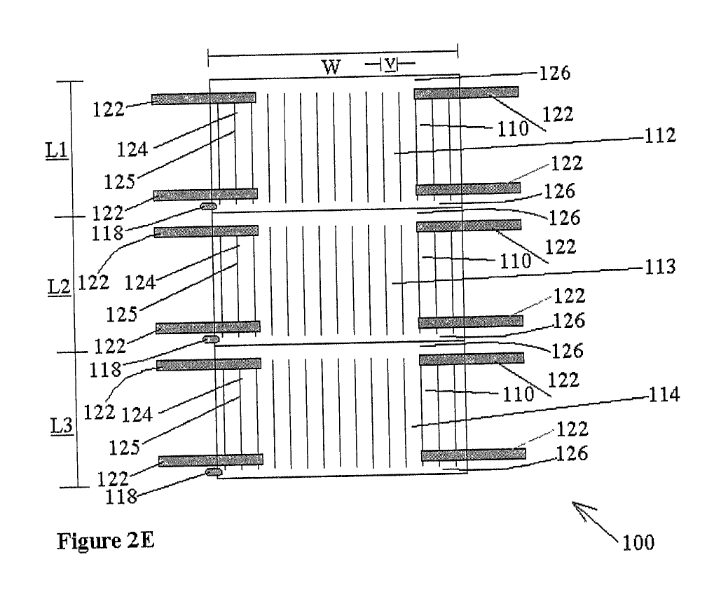

As seen in FIG. 9, device 100 may be adapted for the fixation of a knee.

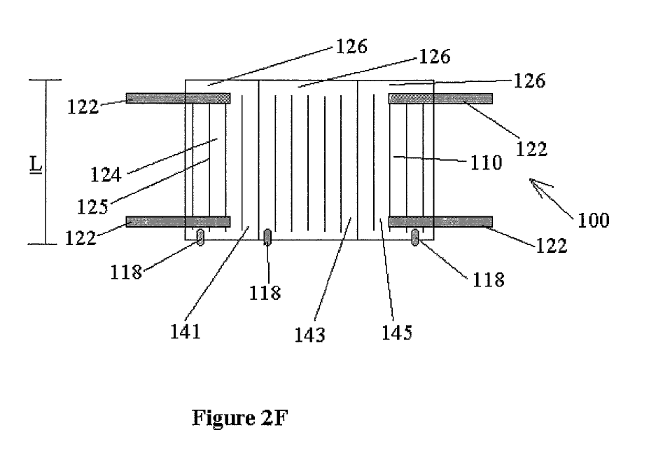

As seen in FIG. 10, device 100 may be adapted for the fixation of a leg.

As seen in FIG. 11, device 100 may be adapted for the fixation of a shoulder.

It will be appreciated that other modes of orthopedic fixation are also possible.

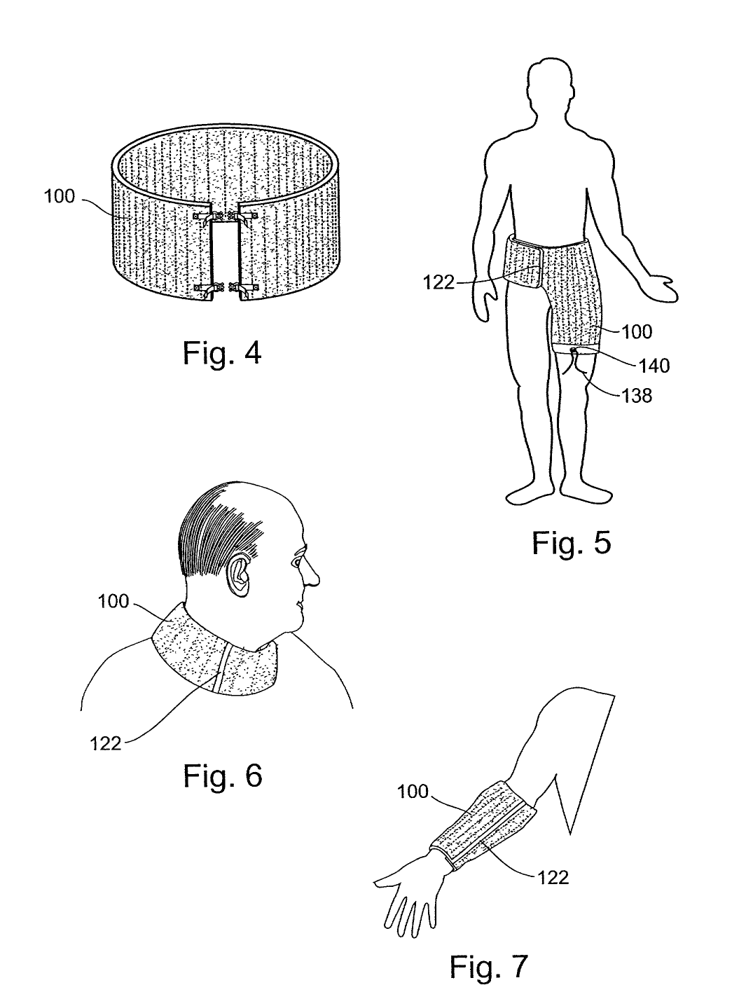

Referring further to the figures, FIG. 12 schematically illustrates adjustable, elastic fixation device 100, designed as an interface between an artificial limb 154 and a stump 152, in accordance with still another preferred embodiment of the present invention. The use of device 100 as an interface with artificial limb 154 is uniquely advantageous because of the adjustable nature of device 100. Generally, with time, there is shrinkage of stump 152. However, device 100 may compensate for that shrinkage, by inflation, for example, as shown in FIGS. 1E, 2B, and 3C-3D.

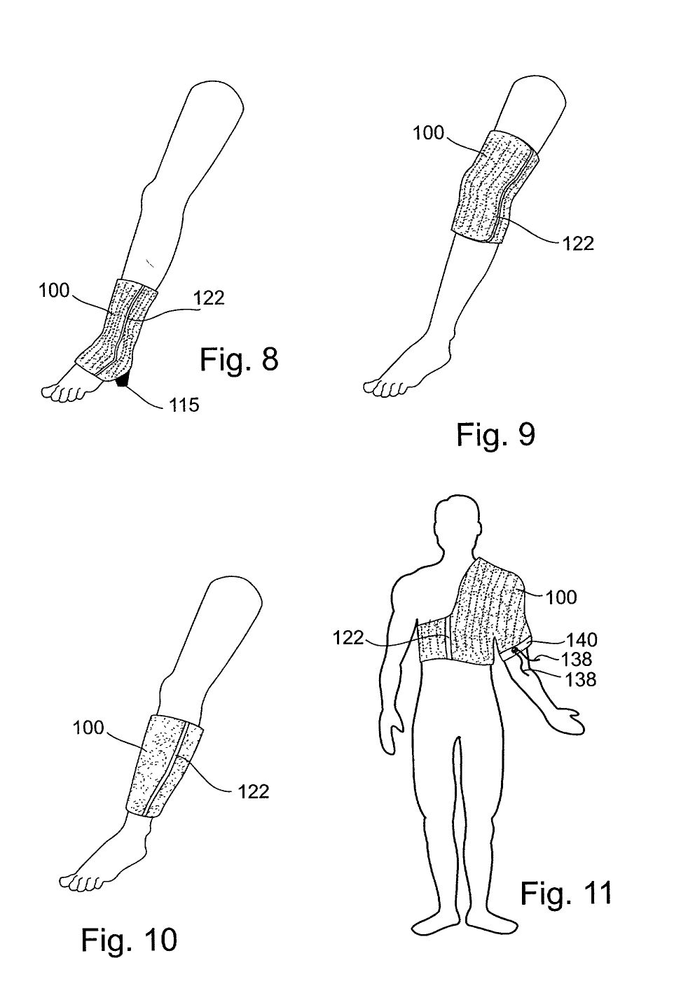

Referring further to the figures, FIGS. 13A-13C schematically illustrates adjustable, elastic fixation device 100 designed as a vest 100, which may be, for example, a combat vest, a bullet proof vest (generally of a rather heavy material), or an outdoor vest, such as a hunting vest, a photography vest, a fishing vest and the like. Alternatively, device 100 may form only a part of or an interface for a vest.

It will be appreciated that at least one adjustable fastener 122 may be designed along the chest of body 120. Alternatively, at least one adjustable fastener 122 may be designed along the back of body 120. Alternatively, at least one adjustable fastener 122 may be designed along one side of body 120. Alternatively, two adjustable fasteners 122 may be designed along two sides of body 120. Alternatively, a combination of the above may be used.

As seen in FIG. 13B, vest 100 may further include at least one, and preferably a plurality of pockets 117 for various pieces of equipment.

As seen in FIG. 13C vest 100 may be a fisherman vest, which further includes a fishing-pole holder 119.

In FIGS. 13A-13C Vest 100 is operative to transfer loads from the spinal column to the pelvis. Additionally, vest 100 takes the load off the shoulders and provides the person wearing it greater freedom of movement.

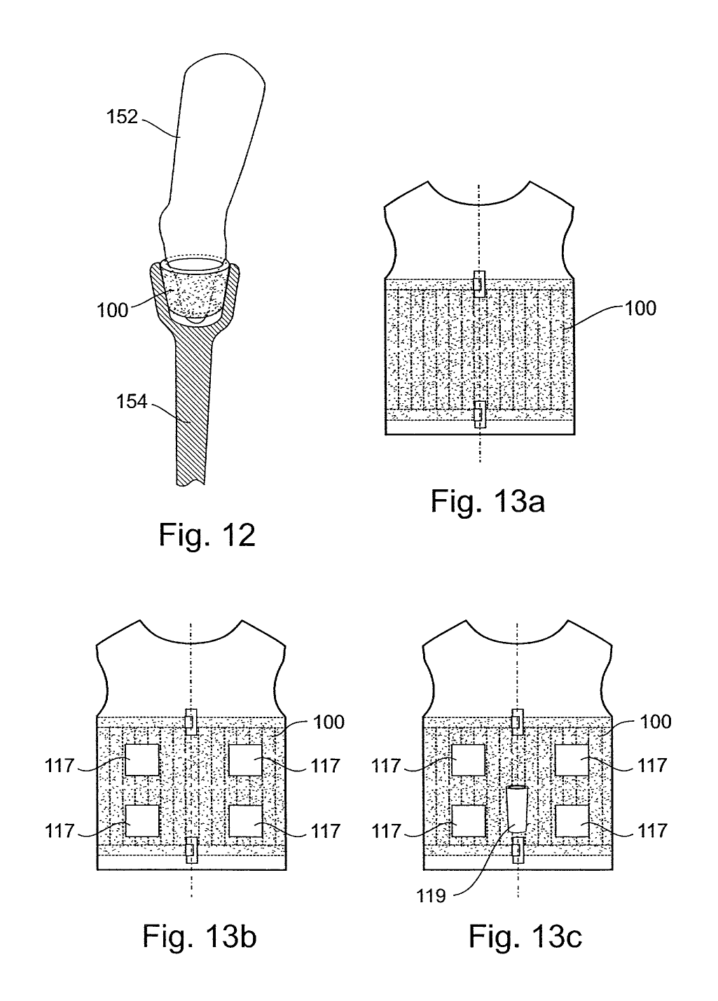

Referring further to the figures, FIG. 14 schematically illustrates adjustable, elastic fixation device 100 adapted for transferring the load of a backpack 162 from the spinal column to the pelvis, in accordance with yet another preferred embodiment of the present invention. While many backpacks in the market include aluminum frames and connecting straps, for improved load distributions, these features add to the weight of the backpack. Additionally, aluminum frames are generally not adjustable. By contrast, device 100 is adjustable and provides for an improved load distribution, with negligible added weight.

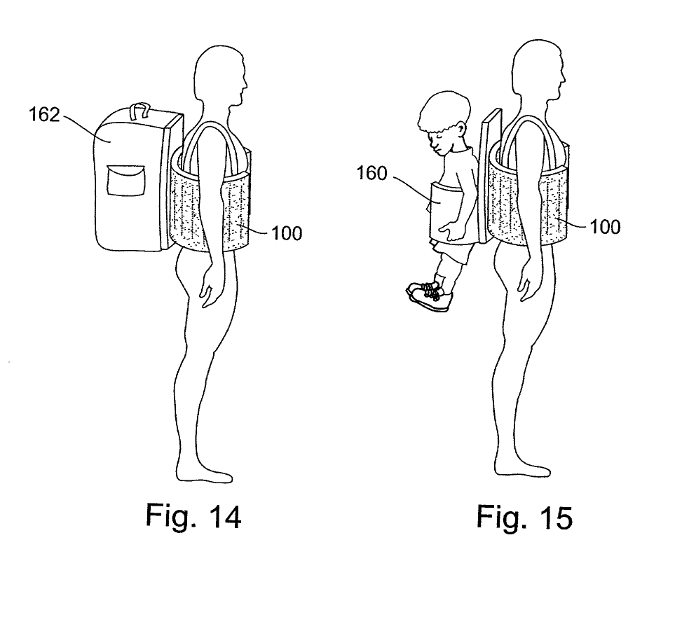

Referring further to the figures, FIG. 15 schematically illustrates adjustable, elastic fixation device 100 designed as a child carrier 160, adapted for transferring the weight of the child from the spinal column to the pelvis, in accordance with yet another preferred embodiment of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An adjustable, elastic fixation device, comprising:
inner and outer thin, flexible, airtight materials, designed to substantially superimpose;
airtight seams, connecting edges of said inner and outer airtight materials, defining an inflatable volume within;
a flexible structure, arranged between said inner and outer materials, for providing a generally even thickness to said inflatable volume, in the inflated state;
a valve, in fluid communication with said inflatable volume, for selectably inflating and selectably deflating said device, as desired;
a controlled inflation device, which may be selectably mounted on said valve, and selectably detached from said valve, for providing gradual inflation to said inflatable volume; and
an adjustable fastener, for adjustably fastening said device on a section of a body.

2. The device of claim 1, wherein when unfastened, said device may open to a generally flat configuration.

3. The device of claim 1, wherein said device is formed as a sleeve.

4. The device of claim 1, and further including a replaceable interface between said inner material, designed on a side of said body, and said body, said replaceable interface being selected from the group consisting of a washable interface and a disposable interface.

5. The device of claim 1, and further including a wrapper, which wraps said inner and outer materials.

6. The device of claim 5, wherein said wrapper is replaceable.

7. The device of claim 1, wherein said flexible structure includes an elastic structure, formed of first and second elastic layers which define repetitive cutout structures thereon, and which are connected by fiber columns.

8. The device of claim 1, wherein said flexible structure includes an elastic structure, formed of coiled springs.

9. The device of claim 1, wherein said flexible structure includes an interconnected channel structure.

10. The device of claim 1, wherein said adjustable fastener includes at least one pair of fastening strips, designed along a length of said device, parallel to a length axis of said body.

11. The device of claim 1, wherein said adjustable fastener includes at least two pairs of fastening strips, designed along a width of said device, perpendicular to a length axis of said body.

12. The device of claim 1, wherein said adjustable fastener includes at least two drawstrings, designed within respective draw channels.

13. The device of claim 1, wherein said device includes a plurality of length sections, each adapted for individual inflation.

14. The device of claim 1, wherein said device includes a plurality of width sections, each adapted for individual inflation.

15. The device of claim 14, wherein each of said width sections is adapted for individual fastening.

16. The device of claim 1, wherein said device is designed as a belt.

17. The device of claim 1, wherein said device is designed as a neck support.

18. The device of claim 1, wherein said device is designed as an arm support.

19. The device of claim 1, wherein said device is designed as a leg support.

20. The device of claim 1, wherein said device is designed as a knee support.

21. The device of claim 1, wherein said device is designed as an ankle support.

22. The device of claim 1, wherein said device is designed as a hip-joint support.

23. The device of claim 1, wherein said device is designed as an interface between a body stump and an artificial limb.

24. The device of claim 1, wherein said device is designed as an interface between a body and a backpack.

25. The device of claim 1, wherein said device is designed as an interface between a body and a child carrier.

26. The device of claim 1, wherein said device is designed as a vest.

27. The device of claim 26, wherein said vest is a combat vest.

28. The device of claim 26, wherein said vest is a bullet-proof vest.

29. The device of claim 26, wherein said vest is an outdoor vest.

30. The device of claim 26, wherein said vest further includes at least one pocket.

31. The device of claim 26, wherein said vest further includes a plurality of pockets.

32. The device of claim 26, wherein said vest further includes a fishing-poll pocket.

33. The device of claim 1, wherein said device further includes at least one first pressure gauge, for monitoring a pressure within said inflatable volume.

34. The device of claim 1, wherein said device further includes at least two first pressure gauges, for monitoring pressures within sections of said inflatable volume.

35. The device of claim 1, wherein said device further includes at least one second pressure gauge, for monitoring a pressure between said device and said body.

36. The device of claim 1, wherein said device further includes at least two second pressure gauges, for monitoring pressures between sections of said device and said body.

37. A method of orthopedic fixation, comprising:
selecting an adjustable, elastic fixation device, of a proper size and mode of fixation, said device comprising:
inner and outer thin, flexible, airtight materials, designed to substantially superimpose;
airtight seams, connecting edges of said inner and outer airtight materials, defining an inflatable volume within;
a flexible structure, arranged between said inner and outer materials, for providing a generally even thickness to said inflatable volume, in the inflated state;
a valve, in fluid communication with said inflatable volume, for selectably inflating and selectably deflating said device, as desired;
a controlled inflation device, which may be selectably mounted on said valve, and selectably detached from said valve, for providing gradual inflation to said inflatable volume; and
an adjustable fastener, for adjustably fastening said device on a section of a body;
fastening said device onto a section of a body, to said desired clearance; and
inflating said inflatable volume, to desired levels of stiffness and tightness.

38. The method of claim 37, and further including correcting said fastening after said inflating, to correct said levels of stiffness and tightness.

39. The method of claim 37, and further including adjusting said fastening with time.

40. The method of claim 37, and further including adjusting said fastening with activity.

41. The method of claim 37, and further including adjusting said inflation with time.

42. The method of claim 37, and further including adjusting said inflation with activity.

43. The method of claim 37, and further including removing and reapplying said device, as needed.

44. The method of claim 37, wherein when unfastened, said device may open to a generally flat configuration.

45. The method of claim 37, wherein said device is formed as a sleeve.

46. The method of claim 37, wherein said device further includes a replaceable interface between said inner material, designed on a side of said body, and said body, said replaceable interface being selected from the group consisting of a washable interface and a disposable interface.

47. The method of claim 37, wherein said device further includes a wrapper, which wraps said inner and outer materials.

48. The method of claim 47, wherein said wrapper is replaceable.

49. The method of claim 37, wherein said flexible structure includes an elastic structure, formed of first and second elastic layers which define repetitive cutout structures thereon, and which are connected by fiber columns.

50. The method of claim 37, wherein said flexible structure includes an elastic structure, formed of coiled springs.

51. The method of claim 37, wherein said flexible structure includes an interconnected channel structure.

52. The method of claim 37, wherein said adjustable fastener includes at least one pair of fastening strips, designed along a length of said device, parallel to a length axis of said body.

53. The method of claim 37, wherein said adjustable fastener includes at least two pairs of fastening strips, designed along a width of said device, perpendicular to a length axis of said body.

54. The method of claim 37, wherein said adjustable fastener includes at least two drawstrings, designed within respective draw channels.

55. The method of claim 37, wherein said device includes a plurality of length sections, each adapted for individual inflation.

56. The method of claim 37, wherein said device includes a plurality of width sections, each adapted for individual inflation.

57. The method of claim 56, wherein each of said width sections is adapted for individual fastening.

58. The method of claim 37, wherein said device is designed as a belt.

59. The method of claim 37, wherein said device is designed as a neck Support.

60. The method of claim 37, wherein said device is designed as an arm support.

61. The method of claim 37, wherein said device is designed as a leg support.

62. The method of claim 37, wherein said device is designed as a knee support.

63. The method of claim 37, wherein said device is designed as an ankle support.

64. The method of claim 37, wherein said device is designed as a hip-joint support.

65. The method of claim 37, wherein said device is designed as an interface between a body stump and an artificial limb.

66. The method of claim 37, wherein said device is designed as an interface between a body and a backpack.

67. The method of claim 37, wherein said device is designed as an interface between a body and a child carrier.

68. The method of claim 37, wherein said device is designed as a vest.

69. The method of claim 68, wherein said vest is a combat vest.

70. The method of claim 68, wherein said vest is a bullet-proof vest.

71. The method of claim 68, wherein said vest is an outdoor vest.

72. The method of claim 68, wherein said vest further includes at least one pocket.

73. The method of claim 68, wherein said vest further includes a plurality of pockets.

74. The method of claim 68, wherein said vest further includes a fishing-poll pocket.

75. The method of claim 37, wherein said device further includes at least one first pressure gauge, for monitoring a pressure within said inflatable volume.

76. The method of claim 37, wherein said device further includes at least two first pressure gauges, for monitoring pressures within sections of said inflatable volume.

77. The method of claim 37, wherein said device further includes at least one second pressure gauge, for monitoring a pressure between said device and said body.

78. The method of claim 37, wherein said device further includes at least two second pressure gauges, for monitoring pressures between sections of said device and said body.
